(19) United States
(12) Patent Application Publication
(10) Pub. No.: US 2023/0074200 A1
(43) Pub. Date: Mar. 9, 2023

(54) METHOD FOR TREATING BOTULINUM TOXIN POISONING

(71) Applicant: Catalyst Pharmaceuticals, Inc., Coral Gables, FL (US)

(72) Inventor: Patrick MCNUTT, Coral Gables, FL (US)

(21) Appl. No.: 17/819,111

(22) Filed: Aug. 11, 2022

Related U.S. Application Data
(60) Provisional application No. 63/234,970, filed on Aug. 19, 2021.

(51) Int. Cl. A61K 31/44; A61P 39/02

(52) U.S. Cl. CPC A61K 31/44; A61P 39/02

(57) ABSTRACT

The present disclosure relates to a method of treating botulism poisoning comprising administering to a subject in need thereof, an effective amount of 3, 4-diaminopyridine, or a pharmaceutically acceptable salt thereof, via continuous infusion, single bolus injection, or orally.

### BACKGROUND

Botulinum neurotoxins (BoNTs) are highly potent poisons produced by the Clostridium genus of anaerobic bacteria. The active neurotoxin is a heterodimer between a 100 kDa heavy chain (HC) and 50 kDa light chain (LC). The HC mediates selective binding to endosomal receptors on the presynaptic membrane of peripheral neurons. Following neuronal uptake via synaptic endocytosis, the LC translocates into the nerve terminal, where it specifically cleaves neuronal soluble N-ethylmaleimide-sensitive factor attachment protein receptor (SNARE) proteins essential for neurotransmitter release. Cleavage of SNARE proteins blocks assembly of core vesicle fusion complexes, preventing vesicle fusion and acetylcholine release. As the concentration of cleaved SNARE proteins increases, motor nerve terminals are unable to reliably elicit muscle contraction, causing muscle weakness that progresses to flaccid paralysis.

Clinical botulism symptoms typically emerge 12-36 h after exposure to BoNT and are caused by peripheral blockade of neurotransmission at neuromuscular junctions and autonomic nerve terminals. At lethal doses, neuroparalytic symptoms manifest as cranial nerve dysfunctions that rapidly advance to life-threatening respiratory weakness. The only clinically approved treatment for botulism is post-exposure prophylaxis with antitoxin, which blocks neuronal uptake of BoNT but has no effect on toxin molecules already bound to or internalized into neurons. Because of the delay between neuronal uptake and toxic manifestations, substantial neuronal uptake can occur before symptoms emerge. Consequently, the majority of symptomatic patients suffering from systemic botulism and treated with antitoxin still require weeks of intensive care support for survival. Since even moderate-scale outbreaks have potential for devastating effects on local healthcare resources, BoNTs are considered Tier 1 select agents by the U.S. government. BoNT serotype A (BoNT/A) is the etiological agent responsible for approximately half of natural botulism cases in the United States and is also the active component in most neurotoxin-based pharmaceuticals. Neurotoxin-based pharmaceuticals, which are injected into muscle for medicinal or cosmetic purposes, can cause off-target effects when overdosed or misplaced.

Off-target effects of localized BoNT injections can include unwanted localized muscle weakness or paralysis near the treatment site. Consequently, treatments for natural and iatrogenic BoNT/A botulism are a high priority.

The stark limitations of antitoxin treatments have necessitated a search for anti-botulism therapies. Considerable effort has been directed to antidotal treatments, primarily small molecule inhibitors (SMIs) that specifically block LC metalloprotease activity inside the nerve terminal. Development of SMIs is complicated by multiple factors, including an expansive substrate-enzyme interface, topologically constrained active site, large degree of conformational flexibility and need for multiple SMIs to block the structurally diverse toxin serotypes. Consequently, no SMIs have been approved to date. Alternatively, intraneuronal delivery of therapeutic antibodies was recently reported to have antidotal efficacy in non-human primates, with physiological reversal of botulism symptoms occurring over days. This delay in therapeutic benefit is consistent with the need to regenerate intact SNARE proteins before symptomatic recovery from botulism and emphasizes that LC inhibitors are unlikely to have acute effects on toxic signs.

Given the limitations of antitoxin and lack of approved intracellular SMIs, there is a critical need for a fast-acting, symptomatic treatment for systemic and localized BoNT poisoning that sustains cholinergic neurotransmission until toxicity recedes.

### BRIEF SUMMARY

The present disclosure relates to the use of 3,4-diaminopyridine and its pharmaceutically acceptable salts in the treatment of botulism. Specifically, the present disclosure relates to methods of treating botulism, wherein the method comprises administering an effective amount of 3,4-diaminopyridine, or a pharmaceutically acceptable salt thereof, via continuous infusion, via injection of a plurality of doses, or via oral administration of a plurality of doses.

In one aspect, the present disclosure provides a method of treating botulism in a subject in need thereof comprising intravenously administering to the subject an effective amount of 3,4-diaminopyridine, or an equivalent amount of a pharmaceutically acceptable salt thereof, in a pharmaceutically acceptable vehicle via continuous infusion.

In some aspects, the effective amount of 3,4-diaminopyridine is infused at a rate of about 0.5 mg/kg of the subject's body weight per hour to about 3 mg/kg of the subject's body weight per hour.

In some aspects, the effective amount of 3,4-diaminopyridine is infused at a rate of about 1.4 mg/kg of the subject's body weight per hour.

In some aspects, the effective amount of 3,4-diaminopyridine is provided in a total daily dose ranging from about 80 mg to about 160 mg of 3,4-diaminopyridine, or the equivalent amount of the pharmaceutically acceptable salt thereof.

In some aspects, the subject is a human subject.

In some aspects, administering the 3,4-diaminopyridine or the equivalent amount of the pharmaceutically acceptable salt thereof, via continuous infusion, achieves a steady-state plasma concentration of about 120 ng/mL of 3,4-diaminopyridine in the subject.

In some aspects, the method comprises administering the total daily dose of 3,4-diaminopyridine or the equivalent amount of the pharmaceutically acceptable salt thereof, via continuous infusion for a plurality of consecutive days.

In some aspects, the botulism is caused by botulinum neurotoxin serotype A.

In some aspects, the botulism comprises localized botulinum neurotoxin intoxication.

In another aspect, the present disclosure provides a method of treating botulism in a subject in need thereof comprising administering to the subject an effective amount of 3,4-diaminopyridine or an equivalent amount of a pharmaceutically acceptable salt thereof, in a pharmaceutically acceptable vehicle via a plurality of single bolus injections.

In some aspects, each of the plurality of single bolus injections comprises about 1 mg to about 3 mg of 3,4-diaminopyridine, or the equivalent amount of the pharmaceutically acceptable salt thereof, per kg of the subject's body weight.

In some aspects, each of the plurality of single bolus injections comprises about 2 mg of 3,4-diaminopyridine, or the equivalent amount of the pharmaceutically acceptable salt thereof, per kg of the subject's body weight.

In some aspects, the effective amount of 3,4-diaminopyridine, or the equivalent amount of the pharmaceutically acceptable salt thereof, is provided in a total daily dose ranging from about 80 mg to about 160 mg of 3,4-diaminopyridine.

In some aspects, the subject is a human subject.

In some aspects, administering the 3,4-diaminopyridine or the equivalent amount of the pharmaceutically acceptable salt thereof, via the plurality of single bolus injections, achieves a steady-state plasma concentration of about 120 ng/mL of 3,4-diaminopyridine in the subject.

In some aspects, the method comprises administering the total daily dose of 3,4-diaminopyridine or the equivalent amount of the pharmaceutically acceptable salt thereof, during each of a plurality of consecutive days.

In some aspects, the botulism is caused by botulinum neurotoxin serotype A.

In some aspects, the botulism comprises localized botulinum neurotoxin intoxication.

In another aspect, the present disclosure provides a method of treating botulism in a subject in need thereof comprising orally administering to the subject an effective amount of 3,4-diaminopyridine phosphate salt.

In some aspects, the effective amount of 3,4-diaminopyridine phosphate salt is provided in a total daily dose equivalent to about 80 mg to about 160 mg of 3,4-diaminopyridine freebase.

In some aspects, the total daily dose of 3,4-diaminopyridine phosphate salt is administered in a plurality of single doses per day.

In some aspects, orally administering the 3,4-diaminopyridine phosphate salt achieves a steady-state plasma concentration of about 120 ng/mL of 3,4-diaminopyridine in the subject.

In some aspects, the subject is a human subject.

In some aspects, the method comprises administering the total daily dose of 3,4-diaminopyridine phosphate salt during each of a plurality of days.

In some aspects, the botulism is caused by botulinum neurotoxin serotype A.

In some aspects, the botulism comprises localized botulinum neurotoxin intoxication.

Additional aspects and advantages of the disclosure will be set forth, in part, in the description that follows, and will flow from the description, or can be learned by practice of the disclosure. The aspects and advantages of the disclosure will be realized and attained by means of the elements and combinations particularly pointed out in the appended claims.

It is to be understood that both the foregoing summary and the following detailed description are exemplary and explanatory only, and are not restrictive of the invention as claimed.

### BRIEF DESCRIPTION OF THE DRAWINGS

FIG. 1A is a graph depicting the rat intravenous LD50 calculated from survival outcomes.

FIG. 1B is a graph depicting the progression of toxic signs in rats challenged with 2.5 LD50 BoNT/A (n=12) over time.

FIG. 1C is a graph depicting the survival curve for rats challenged with 2.5 LD50 BoNT/A (n=12) over time.

FIG. 2A is a graphical depiction of the procedure used for the experiment described in Example 1.

FIG. 2B is a graph depicting the effect of 3,4-DAP treatment on VO₂ in botulinum infected rats.

FIG. 2C is a graph depicting the effect of 3,4-DAP on respiratory rate in botulinum infected rats.

FIG. 2D is a graph depicting the effect of 3,4-DAP treatment on tidal volume in botulinum infected rats.

FIG. 3A depicts a summary of the experimental strategy of the experiment described in Example 1.

FIG. 3B depicts median ±interquartile ratio (IQR) toxic signs for vehicle and 3,4-DAP-treated rats over time for the experiment described in Example 1.

FIG. 3C depicts survival curves for vehicle and 3,4-DAP-treated rats for the experiment described in Example 1.

FIG. 4A is a graphical depiction of the procedure used for the experiment described in Example 2.

FIG. 4B is a graph depicting levels of plasma 3,4-DAP at different infusion dose rates.

FIG. 5A depicts a summary of the experimental strategy of the experiment described in Example 2.

FIG. 5B depicts median ±IQR toxic signs for at the start of infusion for vehicle and 3,4-DAP-treated rats over time for the experiment described in Example 2.

FIG. 5C depicts survival curves for vehicle and 3,4-DAP-treated rats for the experiment described in Example 2.

FIG. 5D depicts median toxic signs over time for vehicle and 3,4-DAP-treated rats for the experiment described in Example 2.

FIG. 5E depicts normalized weights of surviving rats over time for the experiment described in Example 2.

FIG. 5F depicts median ±IQR toxic signs for rats (n=3) from the group infused from 1-5 days with 1.44 mg/kg·h 3,4-DAP for the experiment described in Example 2.

FIG. 6A-6E depict comparisons of diaphragm endplate success rates, endplate potentials (EPPs), miniature EPPs (mEPPs), and quantal content (QC) among the following groups as described in Example 2: BoNT naive rats; rats intoxicated with 110 U/kg BoNT/A and infused with 3,4-DAP from 1-14 days at 0.98 mg/kg·h or 1.44 mg/kg·h and euthanized at 21 days; and rats intoxicated with 110 U/kg BoNT/A and infused with 1.44 mg/kg·h 3,4-DAP from 1-5 days and euthanized.

FIG. 7A-7B is a table (Table 3) providing details on statistical comparisons.

### DETAILED DESCRIPTION

The headings provided herein are not limitations of the various aspects of the disclosure, which can be defined by reference to the specification as a whole. It is also to be understood that the terminology used herein is for the purpose of describing particular aspects only, and is not intended to be limiting, since the scope of the present disclosure will be limited only by the appended claims.

3,4-diaminopyridine, also known as amifampridine or 3,4-DAP, is a potential solution to the critical need for a fast-acting, symptomatic treatment for systemic and localized BoNT poisoning that sustains cholinergic neurotransmission until toxicity recedes. 3,4-DAP has been used as a drug in the treatment of a number of rare muscle diseases, such as congenital myasthenic syndrome. 3,4-DAP is a potassium channel blocker that prolongs action potential duration by reversibly blocking voltage-gated potassium channels, facilitating presynaptic Ca2+influx and increasing acetylcholine release. 3,4-DAP free base form is sold under the trade name Ruzurgi®, which is approved to treat LEMS in pediatric patients. The phosphate salt of 3,4-DAP is sold under the trade name Firdapse® and is approved to treat LEMS in adults.

3,4-DAP has been shown to reverse muscle paralysis in isolated mouse diaphragms poisoned by multiple BoNT serotypes, with particularly efficacy in treatment of serotype A. Short-term treatment with 3,4-DAP has been shown to improve respiratory function and prolong survival in mice at terminal stages of botulism, confirming symptomatic efficacy in vivo. However, 3,4-DAP has a short pharmacodynamic half-life. Prior to the experiments described below with respect to Examples 1 and 2, it remained unknown if repeated or continuous administration of 3,4-DAP could sustain symptomatic benefit until neuromuscular function recovers from botulism paralysis—a process which can take several weeks—while avoiding symptoms of acute neurological toxicity (e.g., seizures) sometimes associated with repeated administration of 3,4-DAP.

### Definitions

For convenience, the meaning of some terms and phrases used in the specification, examples, and appended claims are provided below. Unless stated otherwise, or implicit from context, the following terms and phrases have the meanings provided below. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

The articles “a,” “an,” and “the” are used herein to refer to one or to more than one (i.e., to at least one) of the grammatical object of the article. By way of example, “an element” means one element or more than one element.

As used herein, the term “about” means±10% of the specified value, unless otherwise indicated.

The term “at least” prior to a number or series of numbers is understood to include the number adjacent to the term “at least,” and all subsequent numbers or integers that could logically be included, as clear from context. When “at least” is present before a series of numbers or a range, it is understood that “at least” can modify each of the numbers in the series or range.

As used herein, the terms “comprises,” “comprising,” “having,” “including,” “containing,” and the like are open-ended terms meaning “including, but not limited to.” To the extent a given aspect disclosed herein “comprises” certain elements, it should be understood that present disclosure also specifically contemplates and discloses aspects that “consist essentially of” those elements and that “consist of” those elements.

As used herein the terms “consists essentially of,” “consisting essentially of,” and the like are to be construed as a semi-closed terms, meaning that no other ingredients which materially affect the basic and novel characteristics of an aspect are included.

As used herein, the terms “consists of,” “consisting of,” and the like are to be construed as closed terms, such that an aspect “consisting of” a particular set of elements excludes any element, step, or ingredient not specified in the aspect.

The terms “treat,” “treating,” and “treatment” refer to any indicia of success in the treatment or amelioration of an injury, disease, or condition, including any objective or subjective parameter such as abatement; remission; diminishing of symptoms or making the injury, disease, or condition more tolerable to the patient; slowing in the rate of degeneration or decline; or improving a patient's physical or mental well-being. The treatment or amelioration of symptoms can be based on objective or subject parameters, including the results of a physical examination, neuropsychiatric examinations, or psychiatric evaluation.

The phrase “effective amount” refers to a nontoxic but sufficient amount of the drug or agent to provide the desired effect. The amount that is “effective” will vary from subject to subject, depending on the age and general condition of the individual, the particular active agent or agents, and the like. Thus, it is not always possible to specify an exact “effective amount.” However, an appropriate “effective” amount in any individual case can be determined by one of ordinary skill in the art using routine experimentation. In general, an amount of 3,4-DAP that constitutes an “effective” amount of for treating systemic BoNT intoxication is dependent upon the dose of BoNT that a subject has been exposed to. For example, and without wishing to be bound by a particular theory, a larger total daily dose of 3,4-diaminopyridine may be needed to achieve efficacy in cases where a subject suffering from systemic BoNT intoxication has been exposed to a larger amount of BoNT, whereas a smaller dose of 3,4-diaminopyridine may be effective in cases where a subject has been exposed to a smaller amount of BoNT. It is within the skill of the ordinarily skilled physician to determine the level of exposure to BoNT and to select, or titrate to, the “effective amount” of 3,4-diaminopyridine needed to treat a subject in need thereof, regardless of whether the subject is suffering from systemic or localized BoNT intoxication.

The term “pharmaceutically acceptable salt” refers to salts of a basic compound, such as 3,4-DAP, prepared from pharmaceutically acceptable inorganic and organic acids.

The term “continuous infusion” refers to the administration of a fluid into a subcutaneous space or blood vessel over a prolonged period of time. The prolonged period of time can be any suitable duration from about 1 to about 24 hours, such as 1 hour, 2 hours, 3 hours, or 4 hours; or can be any suitable duration such as from about 1 day to about 21 days, or from about 1 week to about 12 weeks.

The term “botulism” as used herein refers to conditions caused by systemic or localized intoxication with BoNTs of any serotype, such as BoNT/A, including the associated muscular and/or respiratory symptoms regardless of the manner in which the condition is acquired.

The term “toxic sign” as used herein refers to physiological signs associated with botulism. Toxic signs include respiratory signs, such as abdominal paradox and agonal respiratory pattern; and skeletomuscular signs, such as salivation, lethargy, and total body paralysis.

### Methods of Treating BoNT Intoxication

Systemic BoNT intoxication, such as botulism arising from ingesting tainted foods or beverages or from exposure to BoNT, can have serious consequences for afflicted subjects. Without proper treatment, subjects with systemic botulism can suffer from muscle paralysis, respiratory distress, and if left untreated or if treated at too late a stage, death by respiratory collapse.

Likewise, off-target effects resulting from medicinal and/or cosmetic uses of BoNT-based pharmaceuticals (such as for treating conditions including Miege syndrome, migraines, bruxism, facial wrinkles, limb spasticity, spasmodic dysphonia, and others), are of grave concern, particularly if the BoNT-based pharmaceutical is administered in amount beyond the prescribed or approved amount, if the BoNT-based pharmaceutical is administered in the wrong location, or if the BoNT-based pharmaceutical is properly administered but “leaks” into surrounding tissues. Off-target effects of BoNT-based pharmaceuticals include, but are not limited to, unwanted localized muscle weakness or paralysis, ptosis or other muscle drooping, difficulty chewing, and weakness or breathiness of the voice from vocal cord paralysis.

In one aspect, the present disclosure provides a method of treating botulism in a subject in need thereof, comprising intravenously administering to the subject an effective amount of 3,4-diaminopyridine, or an equivalent amount of a pharmaceutically acceptable salt thereof, in a pharmaceutically acceptable vehicle via continuous infusion. Infusion of 3,4-DAP can continue for at least one hour, such as 1-24 hours, or for at least one day, such as 1-21 days, or for at least one week, such as 1-12 weeks.

In some aspects, the effective amount of 3,4-diaminopyridine can be infused at a rate of about 0.1 mg/kg of the subject's body weight per hour to about 5.0 mg/kg of the subject's body weight per hour; such as about 0.5 mg/kg per hour to about 4.5 mg/kg per hour, about 1.0 mg/kg per hour to about 4.0 mg/kg per hour, about 1.5 mg/kg per hour to about 3.5 mg/kg per hour, or about 2.0 mg/kg per hour to about 3.0 mg/kg per hour.

In some aspects, the effective amount of 3,4-diaminopyridine can be infused at a rate of about 0.1 mg/kg of the subject's body weight per hour, about 0.2 mg/kg of the subject's body weight per hour, about 0.3 mg/kg of the subject's body weight per hour, about 0.4 mg/kg of the subject's body weight per hour, about 0.5 mg/kg of the subject's body weight per hour, about 0.6 mg/kg of the subject's body weight per hour, about 0.7 mg/kg of the subject's body weight per hour, about 0.8 mg/kg of the subject's body weight per hour, about 0.9 mg/kg of the subject's body weight per hour, about 1.0 mg/kg of the subject's body weight per hour, about 1.1 mg/kg of the subject's body weight per hour, about 1.2 mg/kg of the subject's body weight per hour, about 1.3 mg/kg of the subject's body weight per hour, about 1.4 mg/kg of the subject's body weight per hour, about 1.5 mg/kg of the subject's body weight per hour, about 1.6 mg/kg of the subject's body weight per hour, about 1.7 mg/kg of the subject's body weight per hour, about 1.8 mg/kg of the subject's body weight per hour, about 1.9 mg/kg of the subject's body weight per hour, about 2.0 mg/kg of the subject's body weight per hour, about 2.1 mg/kg of the subject's body weight per hour, about 2.2 mg/kg of the subject's body weight per hour, about 2.3 mg/kg of the subject's body weight per hour, about 2.4 mg/kg of the subject's body weight per hour, about 2.5 mg/kg of the subject's body weight per hour, about 2.6 mg/kg of the subject's body weight per hour, about 2.7 mg/kg of the subject's body weight per hour, about 2.8 mg/kg of the subject's body weight per hour, about 2.9 mg/kg of the subject's body weight per hour, about 3.0 mg/kg of the subject's body weight per hour, about 3.1 mg/kg of the subject's body weight per hour, about 3.2 mg/kg of the subject's body weight per hour, about 3.3 mg/kg of the subject's body weight per hour, about 3.4 mg/kg of the subject's body weight per hour, about 3.5 mg/kg of the subject's body weight per hour, about 3.6 mg/kg of the subject's body weight per hour, about 3.7 mg/kg of the subject's body weight per hour, about 3.8 mg/kg of the subject's body weight per hour, about 3.9 mg/kg of the subject's body weight per hour, about 4.0 mg/kg of the subject's body weight per hour, about 4.1 mg/kg of the subject's body weight per hour, about 4.2 mg/kg of the subject's body weight per hour, about 4.3 mg/kg of the subject's body weight per hour, about 4.4 mg/kg of the subject's body weight per hour, about 4.5 mg/kg of the subject's body weight per hour, about 4.6 mg/kg of the subject's body weight per hour, about 4.7 mg/kg of the subject's body weight per hour, about 4.8 mg/kg of the subject's body weight per hour, about 4.9 mg/kg of the subject's body weight per hour, or about 5.0 mg/kg of the subject's body weight per hour.

In some aspects the infusion can take place over about 1 to about 24 hours, such as about 2 hours to about 22 hours, about 4 hours to about 20 hours, about 6 hours to about 18 hours, about 8 hours to about 16 hours, or about 10 hours to about 14 hours.

In some aspects, the infusion can take place over about 1 hour, over about 2 hours, over about 3 hours, over about 4 hours, over about 5 hours, over about 6 hours, over about 7 hours, over about 8 hours, over about 9 hours, over about 10 hours, over about 11 hours, over about 12 hours, over about 13 hours, over about 14 hours, over about 15 hours, over about 16 hours, over about 17 hours, over about 18 hours, over about 19 hours, over about 20 hours, over about 21 hours, over about 22 hours, over about 23 hours, over about 24 hours.

In some aspects, the infusion of 3,4-DAP (or its pharmaceutically acceptable salt) can take place at any of the rates described above over more than one day, such as about 3 days to about 21 days, about 5 days to about 19 days, about 7 days to about 17 days, about 9 days to about 15 days, or about 11 days to about 13 days.

In some aspects, the infusion of 3,4-DAP (or its pharmaceutically acceptable salt) can take place at any of the rates described above over about 1 day, over about 2 days, over about 3 days, over about 4 days, over about 5 days, over about 6 days, over about 7 days, over about 8 days, over about 9 days, over about 10 days, over about 11 days, over about 12 days, over about 13 days, over about 14 days, over about 15 days, over about 16 days, over about 17 days, over about 18 days, over about 19 days, over about 20 days, or over about 21 days.

In some aspects, the infusion of 3,4-DAP (or its pharmaceutically acceptable salt) can take place at any of the rates described above over more than one week, such as about 2 weeks to about 12 weeks, about 3 weeks to about 11 weeks, about 4 weeks to about 10 weeks, about 5 weeks to about 9 weeks, or about 6 weeks to about 8 weeks.

In some aspects, the infusion of 3,4-DAP (or its pharmaceutically acceptable salt) can take place at any of the rates described above over about 1 week, over about 2 weeks, over about 3 weeks, over about 4 weeks, over about 5 weeks, over about 6 weeks, over about 7 weeks, over about 8 weeks, over about 9 weeks, over about 10 weeks, over about 11 weeks, or over about 12 weeks.

In some aspects, the effective amount of 3,4-diaminopyridine can be provided in a total daily dose ranging from about 1 mg to about 200 mg of 3,4-diaminopyridine, or an equivalent amount of a pharmaceutically acceptable salt thereof, such as about 5 mg to about 195 mg, about 10 mg to about 190 mg, about 15 mg to about 185 mg, about 20 mg to about 180 mg, about 25 mg to about 175 mg, about 30 mg to about 170 mg, about 35 mg to about 165 mg, about 45 mg to about 160 mg, about 50 mg to about 155 mg, about 55 mg to about 145 mg, about 60 mg to about 140 mg, about 65 mg to about 135 mg, about 70 mg to about 130 mg, about 75 mg to about 125 mg, about 80 mg to about 120 mg, about 85 mg to about 115 mg, about 90 mg to about 110 mg, or about 95 mg to about 100 mg.

In some aspects, the effective amount of 3,4-diaminopyridine can be provided in a total daily dose of about 1 mg, about 5 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, or about 200 mg.

In some aspects, the subject can be a human subject.

In some aspects, administering 3,4-diaminopyridine, or the pharmaceutically acceptable salt thereof, as described herein can result in a 3,4-diaminopyridine steady-state blood plasma concentration of about 70 ng/mL to about 200 ng/mL in the subject, such as about 85 ng/mL to about 195 ng/mL, about 90 ng/mL to about 190 ng/mL, about 95 ng/mL to about 185 ng/mL, about 100 ng/mL to about 180 ng/mL, about 105 ng/mL to about 175 ng/mL, about 110 ng/mL to about 165 ng/mL, about 115 ng/mL to about 160 ng/mL, about 120 ng/mL to about 155 ng/mL, about 125 ng/mL to about 150 ng/mL, or about 130 ng/mL to about 145 ng/mL.

In some aspects, administering 3,4-diaminopyridine as described herein can result in a 3,4-diaminopyridine steady-state blood plasma concentration of about 70 ng/mL, about 75 ng/mL, about 80 ng/mL, about 85 ng/mL, about 90 ng/mL, about 95 ng/mL, about 100 ng/mL, about 105 ng/mL, about 110 ng/mL, about 115 ng/mL, about 120 ng/mL, about 125 ng/mL, about 130 ng/mL, about 135 ng/mL, about 140 ng/mL, about 145 ng/mL, about 150 ng/mL, about 155 ng/mL, about 160 ng/mL, about 165 ng/mL, about 170 ng/mL, about 175 ng/mL, about 180 ng/mL, about 185 ng/mL, about 190 ng/mL, about 195 ng/mL, or about 200 ng/mL.

In some aspects, the method can comprise administering the total daily dose of 3,4-diaminopyridine or an equivalent amount of a pharmaceutically acceptable salt thereof, via continuous infusion for a plurality of consecutive days at any of the rates described herein.

In some aspects, the botulism can be caused by botulinum neurotoxin serotype A.

In another aspect, the present disclosure provides a method of treating botulism in a subject in need thereof, comprising administering to the subject an effective amount of 3,4-diaminopyridine or an equivalent amount of a pharmaceutically acceptable salt thereof, in a pharmaceutically acceptable vehicle via a plurality of single bolus injections.

In some aspects, each of the plurality of single bolus injections comprises about 0.1 mg to about 5.0 mg of 3,4-diaminopyridine, or an equivalent amount of a pharmaceutically acceptable salt thereof, per kg of the subject's body weight, such as about 0.5 mg/kg to about 4.5 mg/kg, about 1.0 mg/kg to about 4.0 mg/kg, about 1.5 mg/kg to about 3.5 mg/kg, or about 2.0 mg/kg to about 3.0 mg/kg.

In some aspects, each of the plurality of single bolus injections can comprise about 0.1 mg of 3,4-diaminopyridine, or an equivalent amount of a pharmaceutically acceptable salt thereof, per kg of the subject's body weight, or about 0.2 mg/kg of the subject's body weight, about 0.3 mg/kg of the subject's body weight, about 0.4 mg/kg of the subject's body weight, about 0.5 mg/kg of the subject's body weight, about 0.6 mg/kg of the subject's body weight, about 0.7 mg/kg of the subject's body weight, about 0.8 mg/kg of the subject's body weight, about 0.9 mg/kg of the subject's body weight, about 1.0 mg/kg of the subject's body weight, about 1.1 mg/kg of the subject's body weight, about 1.2 mg/kg of the subject's body weight, about 1.3 mg/kg of the subject's body weight, about 1.4 mg/kg of the subject's body weight, about 1.5 mg/kg of the subject's body weight, about 1.6 mg/kg of the subject's body weight, about 1.7 mg/kg of the subject's body weight, about 1.8 mg/kg of the subject's body weight, about 1.9 mg/kg of the subject's body weight, about 2.0 mg/kg of the subject's body weight, about 2.1 mg/kg of the subject's body weight, about 2.2 mg/kg of the subject's body weight, about 2.3 mg/kg of the subject's body weight, about 2.4 mg/kg of the subject's body weight, about 2.5 mg/kg of the subject's body weight, about 2.6 mg/kg of the subject's body weight, about 2.7 mg/kg of the subject's body weight, about 2.8 mg/kg of the subject's body weight, about 2.9 mg/kg of the subject's body weight, about 3.0 mg/kg of the subject's body weight, about 3.1 mg/kg of the subject's body weight, about 3.2 mg/kg of the subject's body weight, about 3.3 mg/kg of the subject's body weight, about 3.4 mg/kg of the subject's body weight, about 3.5 mg/kg of the subject's body weight, about 3.6 mg/kg of the subject's body weight, about 3.7 mg/kg of the subject's body weight, about 3.8 mg/kg of the subject's body weight, about 3.9 mg/kg of the subject's body weight, about 4.0 mg/kg of the subject's body weight, about 4.1 mg/kg of the subject's body weight, about 4.2 mg/kg of the subject's body weight, about 4.3 mg/kg of the subject's body weight, about 4.4 mg/kg of the subject's body weight, about 4.5 mg/kg of the subject's body weight, about 4.6 mg/kg of the subject's body weight, about 4.7 mg/kg of the subject's body weight, about 4.8 mg/kg of the subject's body weight, about 4.9 mg/kg of the subject's body weight, or about 5.0 mg/kg of the subject's body weight.

In another aspect, the present disclosure provides a method of treating botulism in a subject in need thereof, comprising orally administering to the subject an effective amount of 3,4-diaminopyridine phosphate salt.

In some aspects, the effective amount of 3,4-diaminopyridine phosphate salt can be provided in a total daily dose equivalent to about 1 mg to about 200 mg of 3,4-diaminopyridine freebase, such as a total daily dose equivalent to about 5 mg to about 195 mg, about 10 mg to about 190 mg, about 15 mg to about 185 mg, about 20 mg to about 180 mg, about 25 mg to about 175 mg, about 30 mg to about 170 mg, about 35 mg to about 165 mg, about 45 mg to about 160 mg, about 50 mg to about 155 mg, about 55 mg to about 145 mg, about 60 mg to about 140 mg, about 65 mg to about 135 mg, about 70 mg to about 130 mg, about 75 mg to about 125 mg, about 80 mg to about 120 mg, about 85 mg to about 115 mg, about 90 mg to about 110 mg, or about 95 mg to about 100 mg.

In some aspects, the effective amount of 3,4-diaminopyridine phosphate salt can be provided in a total daily dose equivalent of about 1 mg, about 5 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, or about 200 mg.

In some aspects, the total daily dose of 3,4-diaminopyridine phosphate salt can be administered in a plurality of single doses per day.

In some aspects, administering 3,4-diaminopyridine phosphate salt as described herein can result in a steady-state 3,4-diaminopyridine blood plasma concentration of about 70 ng/mL to about 200 ng/mL in the subject, such as about 85 ng/mL to about 195 ng/mL, about 90 ng/mL to about 190 ng/mL, about 95 ng/mL to about 185 ng/mL, about 100 ng/mL to about 180 ng/mL, about 105 ng/mL to about 175 ng/mL, about 110 ng/mL to about 165 ng/mL, about 115 ng/mL to about 160 ng/mL, about 120 ng/mL to about 155 ng/mL, about 125 ng/mL to about 150 ng/mL, or about 130 ng/mL to about 145 ng/mL.

In some aspects, administering 3,4-diaminopyridine phosphate salt as described herein can result in a 3,4-diaminopyridine steady-state blood plasma concentration of about 70 ng/mL, about 75 ng/mL, about 80 ng/mL, about 85 ng/mL, about 90 ng/mL, about 95 ng/mL, about 100 ng/mL, about 105 ng/mL, about 110 ng/mL, about 115 ng/mL, about 120 ng/mL, about 125 ng/mL, about 130 ng/mL, about 135 ng/mL, about 140 ng/mL, about 145 ng/mL, about 150 ng/mL, about 155 ng/mL, about 160 ng/mL, about 165 ng/mL, about 170 ng/mL, about 175 ng/mL, about 180 ng/mL, about 185 ng/mL, about 190 ng/mL, about 195 ng/mL, or about 200 ng/mL.

In some aspects, the method can comprise administering the total daily dose of 3,4-diaminopyridine phosphate salt during each of a plurality of days.

It has been surprisingly discovered that continuous infusion with 3,4-DAP or repeating dosing with 3,4-diaminopyridine could reduce toxic signs and sustain survival in subjects suffering from botulism during the acute stage of the illness, which can persist for up to several weeks, without causing symptoms of neurological toxicity. For example, in the experiment described below with respect to Example 2, toxic signs in rat subjects lethally challenged with BoNT resolved after a 14 day course of continuous infusion of 3,4-diaminopyridine. Effective daily doses are discussed elsewhere herein.

The therapeutic efficacy of 3,4-diaminopyridine and its salts in the present methods can be determined by clinicians, for example, by using any suitable assessment. Suitable assessments can include, but are not limited to, analysis of recorded diaphragm endplate potentials, visual observation and analysis of toxic signs, questionnaires of human subjects capable of response, or the like.

### Pharmaceutical Formulations

In some aspects, the present disclosure provide compositions suitable for parenteral administration. For example, 3,4-diaminopyridine can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

When an effective amount of 3,4-diaminopyridine is administered by intravenous, cutaneous, or subcutaneous injection, the composition can be in the form of a pyrogen-free, parenterally acceptable aqueous solution. The preparation of such parenterally acceptable solutions, having due regard to pH, isotonicity, stability, and the like, is within the skill in the art. In some aspects, a composition for intravenous, cutaneous, or subcutaneous injection typically contains an isotonic vehicle.

Pharmaceutical compositions for parenteral administration can include aqueous solutions of 3,4-diaminopyridine . Additionally, suspensions 3,4-diaminopyridine can be prepared as appropriate oily injection suspensions. Suitable lipophilic solvents or vehicles include fatty oils or synthetic fatty acid esters. Aqueous injection suspensions can contain substances that increase the viscosity of the suspension. Optionally, the suspension also can contain suitable stabilizers or agents that increase the solubility of 3,4-diaminopyridine and allow for the preparation of highly concentrated solutions. Alternatively, a composition can be in powder form for constitution with a suitable vehicle, e.g., sterile pyrogen-free water, before use.

3,4-diaminopyridine also can be injected parenterally, for example, intravenously, intramuscularly, subcutaneously, or intracoronarily. For parenteral administration, 3,4-diaminopyridine compositions can be used in the form of a sterile aqueous solution which can contain other substances, for example, salts or monosaccharides, such as mannitol or glucose, to make the solution isotonic with blood.

As an additional aspect, the present disclosure can include kits comprising one or more compounds or compositions packaged in a manner that facilitates their use to practice methods of treating botulism described herein. In one aspect, the kit can include a compound or composition described herein as useful for practice of a method (e.g., a composition comprising 3,4-diaminopyridine), packaged in a container, such as a sealed bottle or vessel, with a label affixed to the container or included in the kit that describes use of the compound or composition to practice the method of the disclosure. In certain aspects, the compound or composition can be packaged in a unit dosage form. The kit further can further include a device suitable for administering the composition according to the intended route of administration, for example, a syringe or drip bag. In another aspect, 3,4-diaminopyridine or its pharmaceutically acceptable salt can be a lyophilate. In this instance, the kit can further comprise an additional container which contains a solution useful for the reconstruction of the lyophilate.

In some aspects, the effective amount of 3,4-diaminopyridine or pharmaceutically acceptable salt thereof can be provided and/or administered in a pharmaceutical formulation suitable for oral administration, such as a tablet formulation of the phosphate salt sold by Catalyst Pharmaceuticals, Inc. under the trademark Firdapse®.

In some aspects, the oral formulation can be selected from the group consisting of a solid, a semi-solid, and a liquid oral formulation. In some aspects, the solid oral formulation can be a tablet, a pill, a dragee, a powder, a granule, or a capsule. Suitable liquid formulations include, for example, aqueous suspensions, solutions, elixirs, and syrups. Suitable semi-solid formulations include, for example, oral gels.

Orally administered pharmaceutical formulations can contain conventional excipients known in the art and can be prepared by conventional methods. Orally administered pharmaceutical formulations of the disclosure can contain one or more pharmaceutically acceptable excipients. Suitable excipients include fillers such as saccharides, for example lactose or sucrose, mannitol, sodium saccharin or sorbitol, magnesiun carbonate, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents can be added such as the above-mentioned starches and also carboxymethyl-starch, cross-linked polyvinyl pyrrolidone, agar, or alginic acid or a salt thereof, such as sodium alginate and sodium starch glycolate. Suitable excipients also include flow-regulating agents and lubricants, for example, silica, talc, stearic acid or salts thereof, such as magnesium stearate or calcium stearate, and/or polyethylene glycol; sweetening agents such as fructose, aspartame or saccharin; flavoring agents such as peppermint, oil of wintergreen, or cherry; coloring agents; and preserving agents, to provide a pharmaceutically palatable preparation. In addition, dye stuffs or pigments can be added to the tablets or dragee coatings, for example, for identification or in order to characterize combinations of active compound doses. Other examples of suitable pharmaceutical excipients are described in Remington's Pharmaceutical Sciences pp. 1447-1676 (Alfonso R. Gennaro ed., 19th ed. 1995), incorporated herein by reference. In one aspect, the excipients are of pharmaceutical grade.

In some aspects, 3,4-diaminopyridine or a salt thereof can be micronized before preparing the oral formulations. Methods known in the art can be used for micronization of 3,4-diaminopyridine or its salts. For example, traditional micronization techniques based on friction to reduce particle size can be used, such as milling, bashing and grinding. A typical industrial mill is composed of a cylindrical metallic drum that usually contains steel spheres. As the drum rotates the spheres inside collide with the particles of the solid, thus crushing them towards smaller diameters. In the case of grinding, the solid particles are formed when the grinding units of the device rub against each other while particles of the solid are trapped in between. Methods like crushing and cutting can also be used for reducing particle diameter. Crushing employs hammer-like tools to break the solid into smaller particles by means of impact. Cutting uses sharp blades to cut the rough solid pieces into smaller ones. In addition, modern micronization methods that use supercritical fluids in the micronization process can be used. These methods use supercritical fluids to induce a state of supersaturation, which leads to precipitation of individual particles. Suitable techniques include the RESS process (Rapid Expansion of Supercritical Solutions), the SAS method (Supercritical Anti-Solvent) and the PGSS method (Particles from Gas Saturated Solutions). These modern techniques allow for greater tuneability of the process. Parameters like relative pressure and temperature, solute concentration, and antisolvent to solvent ratio can be varied to adjust to obtain the desired particle size. The supercritical fluid methods result in finer control over particle diameters, distribution of particle size and consistency of morphology.

In some aspects, micronized 3,4-diaminopyridine or its salt suitable for use in the oral formulations of the present disclosure can be a composition where 90% or more of the particles have a particle size of 20 microns or less (i.e., <20 um). In some aspects, the oral pharmaceutical formulations of the present disclosure comprise micronized 3,4-diaminopyridine phosphate salt. In some aspects, 90% or more of the particles in the micronized 3,4-diaminopyridine phosphate salt have a particle size of 20 microns or less.

### EXAMPLES

The method of treatment described herein is now further detailed with reference to the following examples. These examples are provided for the purpose of illustration only and the aspects described herein should in no way be construed as being limited to these examples. Rather, the aspects should be construed to encompass any and all variations which become evident as a result of the teaching provided herein.

### Example 1

Serial administration of 3,4-DAP prolongs survival following lethal challenge with BoNT/A.

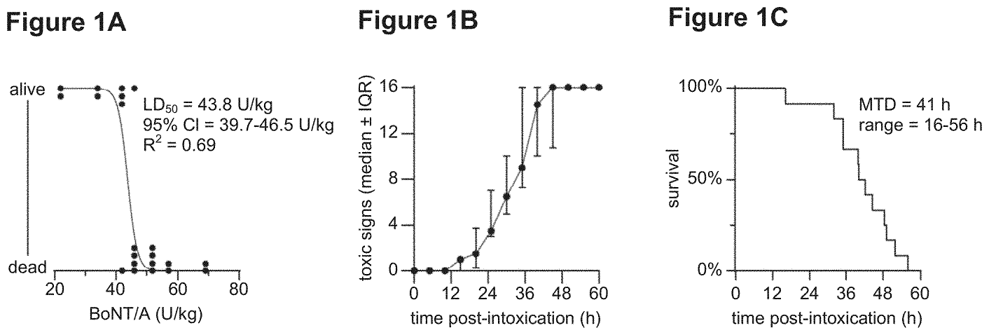

To confirm that 3,4-DAP would reverse botulism toxicity in rats, the effects of repeated 3,4-DAP administration were first evaluated in rats lethally challenged by intravenous administration of 110 U/kg BoNT/A (2.5 LD50; see FIG. 1A).

FIG. 1 generally depicts BoNT/A potency determination and disease progression at 2.5 LD50 in rats. FIG. 1A depicts determination of rat intravenous L1350. Rats were administered 22-69 U/kg BoNT/A by tail vein injection and monitored for survival at 24 h intervals through 7 d. Surviving rats were bright, alert and responsive at 7 d with receding toxic signs of botulism. The LD50 was calculated from survival outcomes using simple linear regression. FIG. 1B depicts the progression of toxic signs in rats challenged with 110 U/kg (2.5 LD50) BoNT/A (n=12). FIG. 1C depicts the survival curve for rats from FIG. 1B. Details on statistical comparisons are presented in FIG. 7A-7B (Table 3).

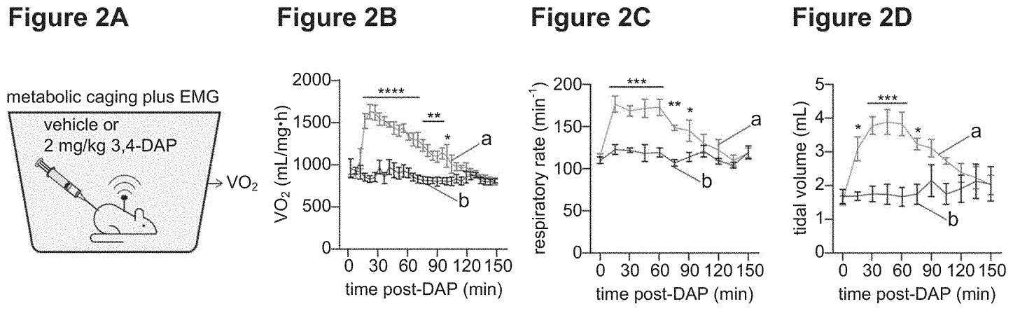

The treatment start time was based on toxic sign manifestation (FIG. 1B) and time-to-death (FIGS. 1C) in toxin potency studies, whereas the treatment interval was estimated from the duration of respiratory effects observed in naive rats treated with 2 mg/kg 3,4-DAP (determined as depicted in FIG. 2). In naive rats, a single dose of <8 mg/kg or 15 consecutive injections of 2 mg/kg 3,4-DAP did not elicit physiological indicators of acute neurological toxicity (see “bolus injection” section of Table 1 below), suggesting this treatment regimen was well-tolerated.

TABLE 1

| | | | behavioral | |
|---|---|---|---|---|
| | gait | | seizure (%), | |
| treatment | change | salivation | max Racine | mortality |
| route | (%) | (%) | score | (%) |

bolus injection (sc)

| | | | | | |
|---|---|---|---|---|---|
| 1 × 1 | mg/kg | 0 | 0 | 0, 0 | 0 |
| 1 × 2 | mg/kg | 0 | 0 | 0, 0 | 0 |
| 1 × 8 | mg/kg | 0 | 0 | 0, 0 | 0 |
| 1 × 16 | mg/kg | 50 | 25 | 0, 0 | 0 |
| 15 × 2 | mg/kg | 0 | 0 | 0, 0 | 0 |

infusion (sc)

| | | | | | |
|---|---|---|---|---|---|
| 0 | mg/kg · h | 0 | 0 | 0, 0 | 0 |
| 0.36 | mg/kg · h | 0 | 0 | 0, 0 | 0 |
| 0.72 | mg/kg · h | 0 | 0 | 0, 0 | 0 |
| 1.44 | mg/kg · h | 0 | 0 | 0, 0 | 0 |

FIG. 2 generally depicts 3,4-DAP pharmacodynamic parameters in naïve rats. Rats implanted with diaphragmatic electrodes and maintained in metabolic caging were administered vehicle saline or 2 mg/kg 3,4-DAP by subcutaneous injection (n=4 per group). FIG. 2A depicts a summary of this experimental strategy. FIGS. 2B-2D depict that 3,4-DAP treatment (trend lines “a”) elicits transient increases in VO₂ (FIG. 2B), respiratory rate (FIG. 2C) and tidal volume (FIG. 2D). Mean values were compared to vehicle (trend lines “b”) at each time point using two-way repeated measures ANOVA followed by Sidak's multiple comparisons test. For FIG. 2B, samples were collected in 5 min bins, whereas for FIG. 2C and FIG. 2D, values were collected at 30 min intervals. ****=p<0.0001; ***=p<0.001; **=p<0.01; and *=p<0.05. Details on statistical comparisons are in FIG. 7A-7B (Table 3).

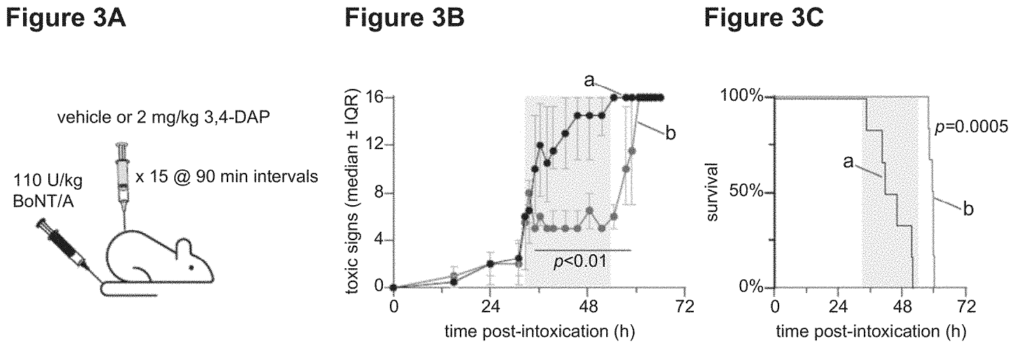

FIG. 3 generally depicts that repeated administration of 3,4-DAP reverses clinical signs of botulism and prolongs survival. Rats were treated with 2 mg/kg 3,4-DAP or saline vehicle in 15 consecutive injections at 90 min intervals, starting 32 h after intoxication (n=6 each; FIG. 3A). In previous studies, 2 mg/kg 3,4-DAP reversed respiratory and skeletal muscle weakness in BoNT/A-intoxicated mice. 3,4-DAP pharmacokinetic properties are similar between rats and mice, suggesting 2 mg/kg 3,4-DAP would also be effective in reversing acute botulism in rats.

FIG. 3B depicts median ±interquartile ratio (IQR) toxic signs for vehicle (trend line “a”) and 3,4-DAP-treated (trend line “b”) rats over time (repeated measures two-way ANOVA with Sidak's multiple comparisons test at each time point). At start of treatment, intoxicated rats exhibited signs of systemic botulism, including abdominal paradox, dysphagia and limb weakness. Compared to vehicle, 3,4-DAP reduced toxic signs within 30 min and sustained symptomatic benefit through the treatment period.

FIG. 3C depicts survival curves for vehicle (trend line “a”) and 3,4- DAP-treated (trend line “b”) rats (Mantel-Cox log-rank test). Gray boxes in panels B and C represent treatment period. Details on statistical comparisons are in FIG. 7A-7B (Table 3). 3,4-DAP significantly increased survival rates from 0% (vehicle) to 100% (3,4-DAP; p=0.002) at the final injection. Toxic signs worsened approximately 1.5 h after cessation of 3,4-DAP treatment, and rats died 2-4 h after the final injection. Collectively, the data depicted in FIGS. 3B and 3C demonstrate robust, albeit transient, effects of repeated bolus injections of 3,4-DAP on toxic signs and survival in rats at terminal stages of botulism.

### Example 2

### Continuous Infusion of 3,4-DAP Reversed Toxic Signs and Enabled Survival.

Summary: 3,4-DAP was continuously infused for 13 days to rats lethally challenged with BoNT serotype A. Clinically relevant doses of 3,4-DAP stabilized toxic signs and enabled survival without adverse effects or re-emergence of toxic signs after treatment withdrawal. Diaphragm endplate recordings from infused rats revealed profound neuromuscular depression at 5 d that was partially reversed in survivors at 21 d, providing a functional mechanism for antidotal efficacy. These data demonstrate strong translational potential for 3,4-DAP in treatment of clinical botulism.

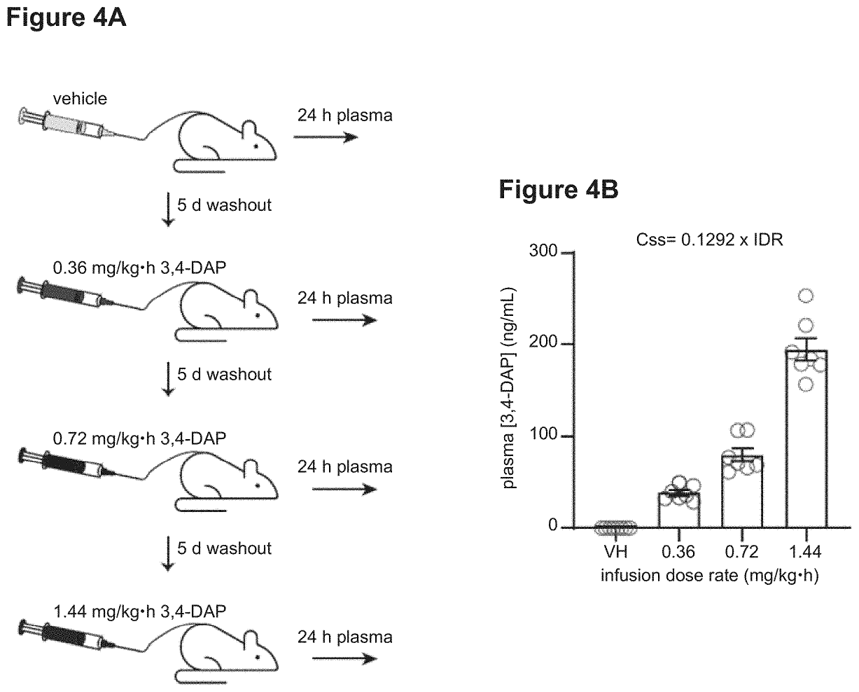

FIG. 4 generally depicts that infusion dose is linearly related to 3,4-DAP steady-state concentrations. To determine whether therapeutic benefits of 3,4-DAP could be sustained over longer periods, 3,4-DAP was continuously infused to lethally intoxicated rats through subcutaneous catheters. First, the steady-state relationship between 3,4-DAP infusion dose and serum concentration was established by measuring 3,4-DAP serum levels after 24 h infusion with 0, 0.36 mg/kg·h, 0.72 mg/kg·h or 1.44 mg/kg·h 3,4-DAP (FIG. 4A; n=7 rats per dose). The relationship between infusion dose rate (IDR) and CSS was determined by linear regression. CSS was linearly related to infusion dose, indicating the absence of saturation effects up to 1.44 mg/kg·h 3,4-DAP (FIG. 4B; the best-fit equation is presented above the graph.). Behavioral signs of neurological toxicity were not observed during infusion at any dose (see “infusion” section of Table 1 above). Details on statistical comparisons are presented in FIG. 7A-7B (Table 3).

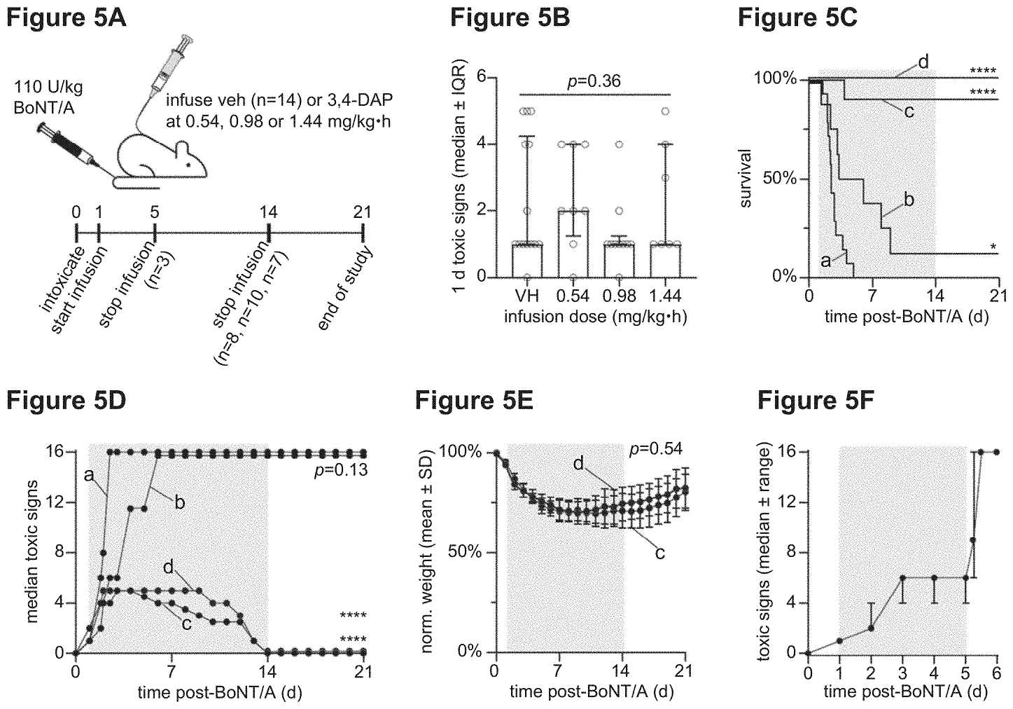

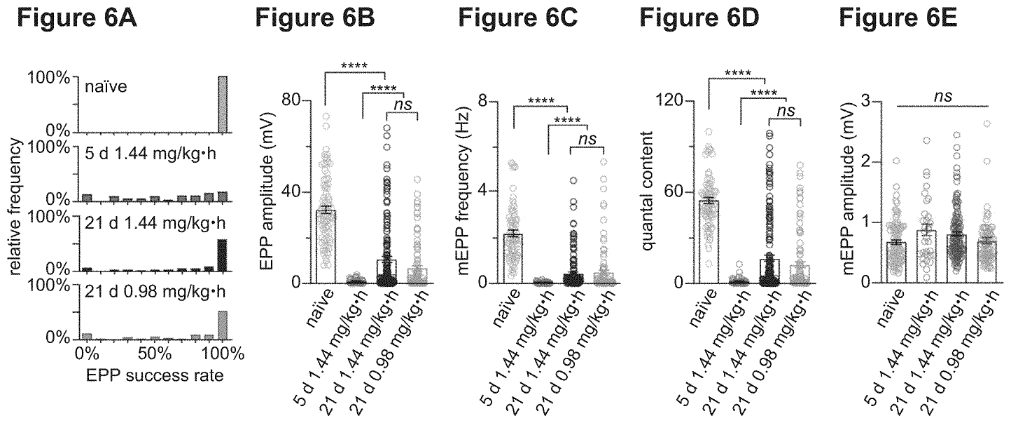

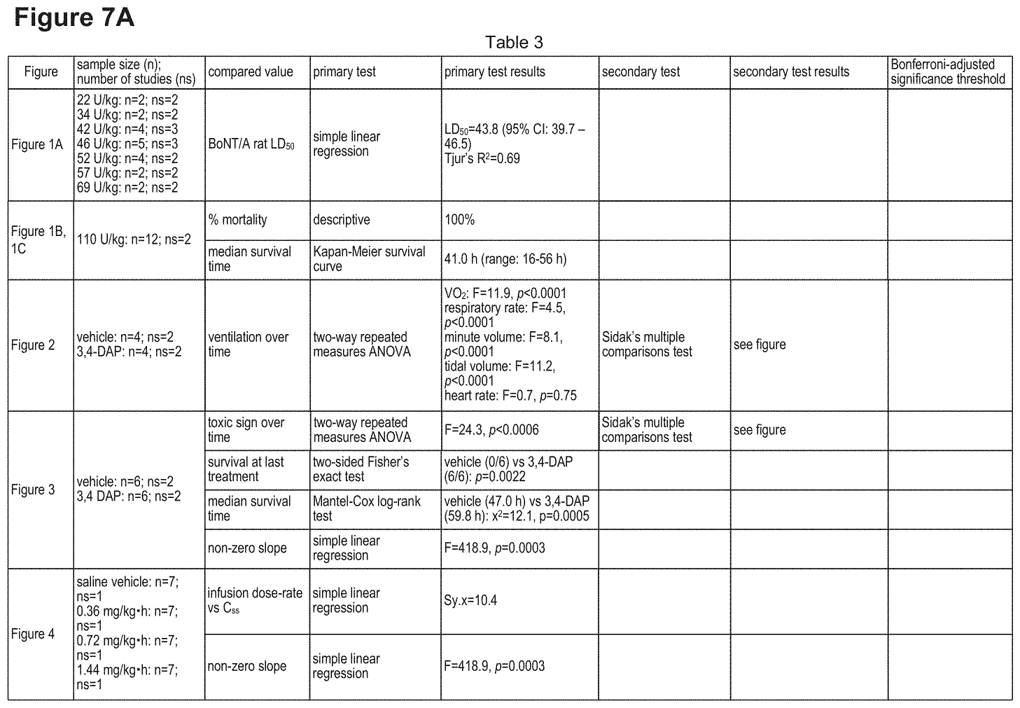

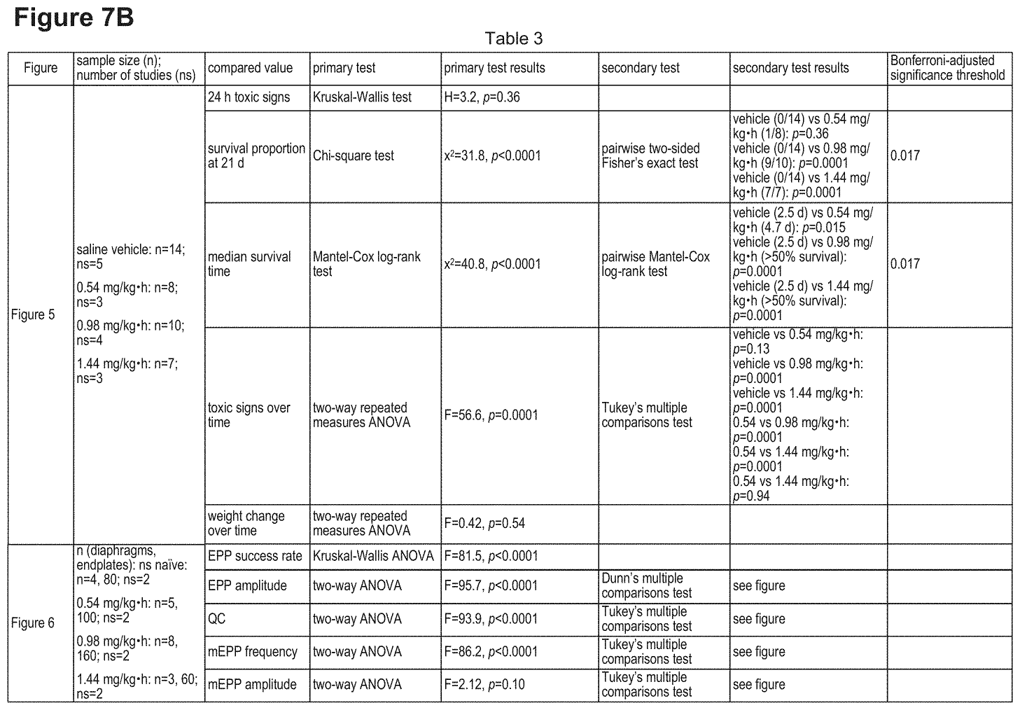

FIG. 5 generally depicts that continuous infusion of 3,4-DAP has both symptomatic and antidotal effects in lethally intoxicated rats. For efficacy studies, rats were lethally intoxicated with 2.5 LD50 BoNT/A and randomized into treatment or vehicle groups Toxic signs, weight, and survival were monitored at 24 h intervals. Gray boxes represent infusion period.

FIG. 5A depicts a summary of the experimental strategy. Catheterized rats were intoxicated by intravenous injection of 110 U/kg BoNT/A. Starting 24-27 h after intoxication, continuous infusion was started with saline vehicle (n=14) or 3,4-DAP at 0.54 mg/kg·h (target CSS=70 ng/mL; n=8), 0.98 mg/kg·h (target CSS=126 ng/mL; n=10) or 1.44 mg/kg·h (target CSS=186 ng/mL; n=8) was started 27 h after intoxication. These infusion doses were intended to generate 3,4-DAP blood levels within the clinical range produced by oral dosing.

FIG. 5B depicts toxic signs at start of infusion (p=0.36; Kruskal-Wallis test), with data presented as median ±IQR. At the start of infusion, 92.3% (36/39) rats exhibited cumulative toxic sign scores >1, with no differences in toxic signs among groups.

FIG. 5C depicts survival curves for each treatment group (Mantel-Cox log-rank test; overall p<0.0001). Trend line “a” corresponds to the saline-vehicle group. Trend line “b” corresponds to the group administered 3,4-DAP at 0.54 mg/kg·h. Trend line “c” corresponds to the group administered 3,4-DAP at 0.98 mg/kg·h Trend line “d” corresponds to the group administered 3,4-DAP at 1.44 mg/kg·h. These designations are consistent through panels C-E. Significance indicators indicate pairwise comparisons made to the saline-vehicle vehicle group. The median survival time of vehicle-infused rats was 2.5 d (range: 2.0-4.5 d). Among all treatment conditions, infusion with 3,4-DAP improved survival proportions (p<0.0001) and median survival time (p<0.0001) compared to vehicle. Pairwise analyses revealed a clear dose-dependent effect of 3,4-DAP infusion rate on survival. Although treatment with 0.54 mg/kg·h 3,4-DAP nearly doubled median survival time versus vehicle (4.7 d; p=0.015), only 12.5% of rats survived to 21 d (1/8; p=0.36 versus vehicle). Infusion with 0.98 mg/kg·h or 1.44 mg/kg·h 3,4- DAP produced 90% survival (p<0.0001 versus vehicle) and 100% survival (p<0.0001 versus vehicle), respectively. Equivalent human total daily doses can be about 1-200 mg/day, for a target CSS of at least about 120 ng/mL. A summary of outcomes for the continuously-infused rats is depicted below in Table 2.

TABLE 2

| infusion | target | | | P | median | | P |
|---|---|---|---|---|---|---|---|
| dose rate | Css | % | | value | survival | | value |
| (mg/kg · | (ng/ | survival | | vs ve- | days | | vs ve- |
| h) | mL) | (ratio) | | hicle | [range] | | hicle |
| 0 | 0 | 0% | (0/13) | — | 2.5 | [1.5-5.0] | — |
| 0.54 | 70 | 12.5% | (1/8) | 0.36 | 4.7 | [1.4-9.0] | 0.015 |

| | | | | | | |
|---|---|---|---|---|---|---|
| 0.98 | 126 | 90% | (9/10) | <0.0001 | undefined | <0.0001 |
| 1.44 | 186 | 100% | (7/7) | <0.0001 | undefined | <0.0001 |

FIG. 5D depicts median toxic signs for each group over time (two-way ANOVA with Tukey's; overall p<0.0001). Significance indicators indicate pairwise comparisons made to vehicle. Toxic signs stabilized by 2 d in surviving rats as moderate abdominal paradox, limb weakness and salivation, and resolved by 14 d, at which time infusion was stopped. Toxic signs were improved by infusion with 0.98 mg/kg·h or 1.44 mg/kg·h 3,4-DAP compared to vehicle (p<0.0001), indicating continuous infusion had sustained symptomatic benefit.

FIG. 5E depicts mean±SD normalized weights for survivors in 0.98 mg/kg·h and 1.44 mg/kg·h treatment groups (two-way ANOVA; p=0.54). Following cessation of infusion, surviving rats remained active, alert and responsive, without symptomatic rebound through 21 d (FIG. 5D) and with gradual weight recovery (FIG. 5E). Video recordings showed that 3,4-DAP-infused rats were active 21 d after challenge with 2.5 LD50 BoNT/A. Video recordings were collected at 21 d after BoNT/A intoxication and were representative of rat activities. Surviving rats showed high levels of activity, including grooming, exploratory behaviors and chow consumption.

FIG. 5F depicts median ±IQR toxic signs for rats (n=3) infused from 1-5 d with 1.44 mg/kg·h 3,4-DAP. Treatment was withdrawn at 5 d and toxic signs were monitored at 6 h intervals. ****=p<0.0001, *=p<0.05. Details on statistical comparisons are in FIG. 7A-7B (Table 3). To determine whether antidotal effects required continuous infusion of 3,4-DAP, BoNT/A intoxicated rats (n=3) were infused with 1.44 mg/kg·h 3,4-DAP from 1-5 d and then switched to saline vehicle. The 5 d time point was chosen because (1) 100% of vehicle-infused rats were deceased by 5 d, indicating the decline in respiratory function below the level sufficient for survival, and (2) toxic signs reached a maximum between 3-5 d in 3,4-DAP-infused survivors, suggesting that paralysis reached peak severity. Following cessation of 3,4-DAP infusion, toxic signs worsened within 3-6 h and all rats were deceased within 12 h. The need for continuous treatment with 3,4-DAP beyond 5 d suggested that antidotal efficacy resulted from sustained symptomatic benefits.

Survivors of lethal BoNT/A challenge exhibited significant recovery of neurophysiological function from 5 to 21 d.

FIG. 6 generally depicts the time-dependent recovery of diaphragm endplate potentials in 3,4-DAP infused intoxicated rats. To identify mechanisms contributing to the recovery of respiratory function in 3,4-DAP-treated rats, evoked endplate potentials (EPPs) and spontaneous miniature endplate potentials (mEPPs) were recorded from diaphragm muscle fibers and compared among naïve rats, surviving rats euthanized 21 d after BoNT/A challenge (7 d after withdrawal of 3,4-DAP infusion), and rats euthanized 5 d after BoNT/A challenge (4 d after start of infusion with 1.44 mg/kg·h 3,4-DAP; FIG. 3A). Phrenic nerve stimulation with a train of 10 impulses at 0.2 Hz produced EPPs with 100% success rate in naïve rats (FIG. 3A).

FIGS. 6B-6E depict mean±SEM scatter dot plots for (B) EPP amplitudes, (C) mEPP frequencies, (D) QC and (E) mEPP amplitudes. For B-E, means were compared using two-way ANOVA followed by Tukey's multiple comparisons test. ****=p<0.0001. Details on statistical comparisons are in FIG. 7A-7B (Table 3). Following BoNT/A treatment, the mean EPP success rate declined to 60.5±5.3% at 5 d (p<0.0001 vs naïve) and recovered to 75.4±3.5% (0.98 mg/kg·h; p<0.0001 vs 5 d) and 79.7±2.5% (1.44 mg/kg·h; p<0.0001 vs 5 d) at 21 d. Similarly, mean EPP amplitude (FIG. 3B), miniature EPP (mEPP) frequency (FIG. 3C) and quantal content (QC; FIG. 3D), which estimates the number of vesicles that fuse during each nerve stimulus, were significantly depressed at 5 d and partially recovered at 21 d. In contrast, mEPP amplitudes did not change after intoxication, consistent with findings that BoNT reduces mEPP frequency but not quantal size (FIG. 6E). The 0.98 mg/kg·h and 1.44 mg/kg·h treatment groups showed no differences in EPP amplitude (p>0.99), mEPP frequency (p>0.99) or QC (p>0.99), indicating that infusion of 3,4-DAP from 1-14 d did not have dose-dependent effects on recovery of neurotransmission at 21 d. That is, 3,4-DAP infusion sustained survival at times when phrenic neurotransmission was profoundly depressed.

### DISCUSSION

The examples described herein demonstrate that continuous infusion of 3,4-DAP from 1-14 d has dose-dependent symptomatic and antidotal efficacy in rats challenged with 2.5 LD50 BoNT/A. Rats remained asymptomatic after treatment withdrawal, despite neurophysiological evidence of residual impairment at diaphragm motor endplates. Collectively, these data demonstrate infusion of human-equivalent doses of 3,4-DAP has sustained symptomatic benefits that can allow survival from lethal botulism exposures.

During development of a continuous infusion system to prolong the pharmacodynamic effects of 3,4-DAP in treatment of botulism toxemia, it was determined that 3,4-DAP infusion rate could be tuned to maximize symptomatic benefits while avoiding high-dose neurological effects and inter-dose symptomatic breakthrough. There was no evidence of functional toxicity or loss of efficacy throughout the infusion period, suggesting that continuous infusion for 13 d can be well-tolerated. By extension, the sustained efficacy of 3,4-DAP in treating botulism suggests that continuous infusion can also be effective in treatment of LEMS patients.

In comparison to the profound reduction in neurotransmission observed in 5 d endplate recordings, neurotransmission was significantly improved in diaphragms isolated from survivors at 21 d, albeit still depressed compared to naïve rats. These data indicate (1) the symptomatic benefits of >0.98 mg/kg·h 3,4-DAP on cholinergic neurotransmission are sufficient to sustain survival at otherwise lethal time points and (2) synaptic function is sufficiently recovered by 14 d to support survival after 3,4-DAP withdrawal. Nevertheless, these data clearly illustrate profound acute depression of neurotransmission at 5 d and significant recovery at 21 d in surviving rats after lethal challenge with BoNT/A.

Symptomatic foodborne botulism cases typically require artificial ventilation for a median of 1.5-2 weeks. The primary clinical benefit of post-symptomatic antitoxin administration is reduced duration of disease, suggesting antitoxin decreases intracellular toxin load and, thus, the severity of intoxication. Because 3,4-DAP efficacy is inversely related to the severity of intoxication, symptomatic treatment with 3,4-DAP can be expected to be more effective in reversing botulism symptoms in patients treated with antitoxin. 3,4-DAP works orthogonally to antitoxin, and the risks of adverse drug interactions are low. Multimodal therapy with antitoxin and 3,4-DAP can offer several advantages over monotherapy with either drug by accelerating recovery from botulism, reducing the risk of life-threatening hospital-acquired diseases, decreasing treatment costs and freeing limited resources for other critical patients. In combination with an intracellular antidote that clears or inactivates the BoNT light chain, this multimodal therapy can be a comprehensive treatment strategy for botulism. Moreover, the majority of botulism patients continue to exhibit neurological deficits and muscle weakness after discharge. Although the pathophysiologies responsible for persistent symptoms remain unknown, they can be related to depressed neurotransmission (as suggested by FIG. 3), and likewise susceptible to 3,4-DAP treatment.

Previous efforts to understand the toxic mechanisms of BoNT on neurotransmission have relied on either supraphysiological intoxication of isolated diaphragm muscle preparations or local administration of paralytic doses to skeletal muscles, neither of which recapitulate the toxicokinetics of a systemic lethal challenge. This was the first animal model in which changes in neurotransmission in response to a lethal, systemic botulism challenge were functionally correlated to physiological metrics during recovery.

Although botulism cases usually involve less than 5 LD50, in rare cases exposures can exceed 100 LD50. Without wishing to be bound by a particular theory, it is believed that 3,4- DAP can be less effective in more severely paralyzed muscles, suggesting 3,4-DAP can have reduced efficacy in cases involving higher BoNT doses. However, such reduced efficacy can be at least partially ameliorated as neuromuscular junction repair progresses, thereby reducing total recovery time.

In conclusion, continuous infusion with 3,4-DAP had both symptomatic and antidotal effects in rats challenged with lethal doses of BoNT/A. Therapeutic benefits emerged at 3,4-DAP exposure levels produced by standard clinical dosing. Survival required continuous 3,4-DAP infusion beyond 5 d, suggesting antidotal outcomes emerged from sustained symptomatic effects. These data illustrate strong translational potential for 3,4-DAP as a treatment of clinical botulism caused by the serotype most commonly associated with human disease.

Having now fully described this disclosure, it will be understood by those of ordinary skill in the art that the same can be performed within a wide and equivalent range of conditions, formulations, and other parameters without affecting the scope of the invention or any aspect thereof.

Other aspects of the disclosure will be apparent to those skilled in the art from consideration of the specification and practice of the invention disclosed herein. It is intended that the specification be considered exemplary only, with a true scope and spirit of the invention being indicated by the following claims.

All patents, patent applications, and publications cited herein are fully incorporated by reference herein in their entirety.

1. A method of treating botulism in a subject in need thereof, comprising intravenously administering to the subject an effective amount of 3,4-diaminopyridine, or an equivalent amount of a pharmaceutically acceptable salt thereof, in a pharmaceutically acceptable vehicle via continuous infusion.

2. The method of claim 1, wherein the effective amount of 3,4-diaminopyridine is infused at a rate of about 0.5 mg/kg of the subject's body weight per hour to about 3 mg/kg of the subject's body weight per hour.

3. The method of claim 2, wherein the effective amount of 3,4-diaminopyridine is infused at a rate of about 1.4 mg/kg of the subject's body weight per hour.

4. The method of claim 1, wherein the effective amount of 3,4-diaminopyridine is provided in a total daily dose ranging from about 80 mg to about 160 mg of 3,4-diaminopyridine, or the equivalent amount of the pharmaceutically acceptable salt thereof.

5. The method of claim 4, wherein the subject is a human subject.

6. The method of claim 1, wherein the administration of 3,4-diaminopyridine or the equivalent amount of the pharmaceutically acceptable salt thereof, achieves a steady-state plasma concentration of about 120 ng/mL of 3,4-diaminopyridine in the subject.

7. The method of claim 1, comprising administering the total daily dose of 3,4-diaminopyridine or the equivalent amount of the pharmaceutically acceptable salt thereof, via continuous infusion for a plurality of consecutive days.

8. The method of claim 1, wherein the botulism is caused by botulinum neurotoxin serotype A.

9. The method of claim 1, wherein the botulism comprises localized botulinum neurotoxin intoxication.

10. A method of treating botulism in a subject in need thereof, comprising administering to the subject an effective amount of 3,4-diaminopyridine or an equivalent amount of a pharmaceutically acceptable salt thereof, in a pharmaceutically acceptable vehicle via a plurality of single bolus injections.

11. The method of claim 10, wherein each of the plurality of single bolus injections comprises about 1 mg to about 3 mg of 3,4-diaminopyridine, or the equivalent amount of the pharmaceutically acceptable salt thereof, per kg of the subject's body weight.

12. The method of claim 11, wherein each of the plurality of single bolus injections comprises about 2 mg of 3,4-diaminopyridine, or the equivalent amount of the pharmaceutically acceptable salt thereof, per kg of the subject's body weight.

13. The method of claim 10, wherein the effective amount of 3,4-diaminopyridine, or the equivalent amount of the pharmaceutically acceptable salt thereof, is provided in a total daily dose ranging from about 80 mg to about 160 mg of 3,4-diaminopyridine.

14. The method of claim 13, wherein the subject is a human subject.

15. The method of claim 13, wherein the administration of 3,4-diaminopyridine or the equivalent amount of the pharmaceutically acceptable salt thereof, achieves a steady-state plasma concentration of about 120 ng/mL of 3,4-diaminopyridine in the subject.

16. The method of claim 10, comprising administering the total daily dose of 3,4-diaminopyridine or the equivalent amount of the pharmaceutically acceptable salt thereof, during each of a plurality of consecutive days.

17. The method of claim 10, wherein the botulism is caused by botulinum neurotoxin serotype A.

18. The method of claim 10, wherein the botulism comprises localized botulinum neurotoxin intoxication.

19. A method of treating botulism in a subject in need thereof, comprising orally administering to the subject an effective amount of 3,4-diaminopyridine phosphate salt.

20. The method of claim 19, wherein the effective amount of 3,4-diaminopyridine phosphate salt is provided in a total daily dose equivalent to about 80 mg to about 160 mg of 3,4-diaminopyridine freebase.

21. The method of claim 19, wherein the total daily dose of 3,4-diaminopyridine phosphate salt is administered in a plurality of single doses per day.

22. The method of claim 19, wherein the administration of 3,4-diaminopyridine phosphate salt achieves a steady-state plasma concentration of about 120 ng/mL of 3,4-diaminopyridine in the subject.

23. The method of claim 19, wherein the subject is a human subject.

24. The method of claim 19, comprising administering the total daily dose of 3,4-diaminopyridine phosphate salt during each of a plurality of days.

25. The method of claim 19, wherein the botulism is caused by botulinum neurotoxin serotype A.

26. The method of claim 19, wherein the botulism comprises localized botulinum neurotoxin intoxication.
